(19) United States
(12) Plant Patent Application Publication
(10) Pub. No.: US 2015/0257325 P1
(43) Pub. Date: Sep. 10, 2015

(54) Euphorbia milii plant named '12-01-ZEU'

(50) ;

(71) Applicant: Steen Thomsen, Sonderso (DK)

(72) Inventor: Steen Thomsen, Sonderso (DK)

(73) Assignee: Hjortebjerg Plant License, Sonderso (DK)

(21) Appl. No.: 14/544,905

(22) Filed: Mar. 6, 2015

(30) Foreign Application Priority Data
Mar. 7, 2014 (QZ) 201403293

(51) Int. Cl. A01H 5/00

(52) U.S. Cl. PLT/302

(57) ABSTRACT

A new and distinct cultivar of Euphorbia milii plant named ‘12-01-ZEU,’ particularly characterized by its medium plant height and width; erect, compact growth habit; grouped disposition of spines; leaves of medium width with the color of the upper side being dark green and under side being dark red; and upper side color of its flowers being RHS 045D.

### LATIN NAME OF THE GENUS AND SPECIES OF THE PLANT CLAIMED

Euphorbia milii Desmoul.

### VARIETY DENOMINATION

‘12-01 -ZEU. ’

### CROSS REFERENCE TO RELATED APPLICATIONS

This application claims priority under 35 U.S.C. §119(f) of the Community Plant Variety Office (CPVO) application No. 201403293 filed Mar. 7, 2014.

### BACKGROUND OF THE INVENTION

The present invention relates to a new and distinct cultivar of Euphorbia plant, botanically known as Euphorbia milii Desmoul., commonly known by the name of Crown of Thorns, and hereinafter referred to by the name ‘12-01-ZEU.’

The new Euphorbia cultivar is a product of a planned breeding program conducted by the inventor, Steen Thomsen, in Hjortebjergvej, Denmark. The new Euphorbia cultivar originated from selection from random crossing of proprietary seedlings of Euphorbia milii cultivars. The inventor selected the new Euphorbia milii cultivar as a single flowering plant from the progeny of the above polycrosses on the basis of the color of its flowers and leaves.

Asexual reproduction of the new cultivar by terminal cuttings taken and propagated in trial production batches in Hjortebjergvej, Denmark, has demonstrated that the unique features of this new Euphorbia cultivar are stable and reproduce true-to-type in many successive generations of asexual reproduction.

### BRIEF SUMMARY OF THE INVENTION

The following traits have been repeatedly observed and are determined to be the unique characteristics of ‘12-01-ZEU.’ These characteristics in combination distinguish ‘12-01-ZEU’ as a new and distinct Euphorbia cultivar:
1. Plant height of about 20-22 cm and plant width of about 16-18 cm.
2. Grouped disposition of spines.
3. Leaf width about 2 cm.
4. Leaf color on the upper side being dark green and on the under side being dark red.
5. The color of the upper side of its flowers is RHS 045D
6. Erect, compact growth habit.

Plants of the cultivar ‘12-01-ZEU’ can be compared to plants of the Euphorbia milii cultivar ‘HJOR 048’ (patented, U.S. Plant Pat. No. 17,605). In side-by-side comparisons conducted by the inventor in Denmark, plants of the new cultivar ‘12-01-ZEU’ and the comparison cultivar ‘HJOR 048’ differ in the following characteristics:
1. Plants of ‘12-01-ZEU’ have flowers of color RHS 045D, whereas plants of ‘HJOR 048’ have flowers of color RHS 054A.
2. Plants of ‘12-01-ZEU’ have leaves with upper side color being dark green, whereas plants of ‘HJOR 048’ have leaves with upper side color being green.
3. Plants of ‘12-01-ZEU’ have leaves of about 5-6 cm in length and about 1.5-2 cm in width, whereas plants of ‘HJOR 048’ have leaves of about 10 cm in length and about 4 cm in width.
4. Plants of ‘ 12-01-ZEU’ produce about 20-30 flowers and buds per plant, whereas plants of ‘HJOR 048’ produce about 100 to 120 flowers and buds per plant.

### BRIEF DESCRIPTION OF THE DRAWINGS

The accompanying color photographs illustrate the overall appearance and details of flower form, color and structures of the new cultivar ‘12-01-ZEU’, showing the colors as true as it is reasonably possible to obtain in color reproductions of this type. Colors in the photographs may differ slightly from the color values cited in the detailed botanical description, which more accurately describe the actual colors of the new Euphorbia cultivar.

FIG. 1 shows a top view of a typical flowering plant of ‘12-01-ZEU’ as grown in an 11 cm pot.

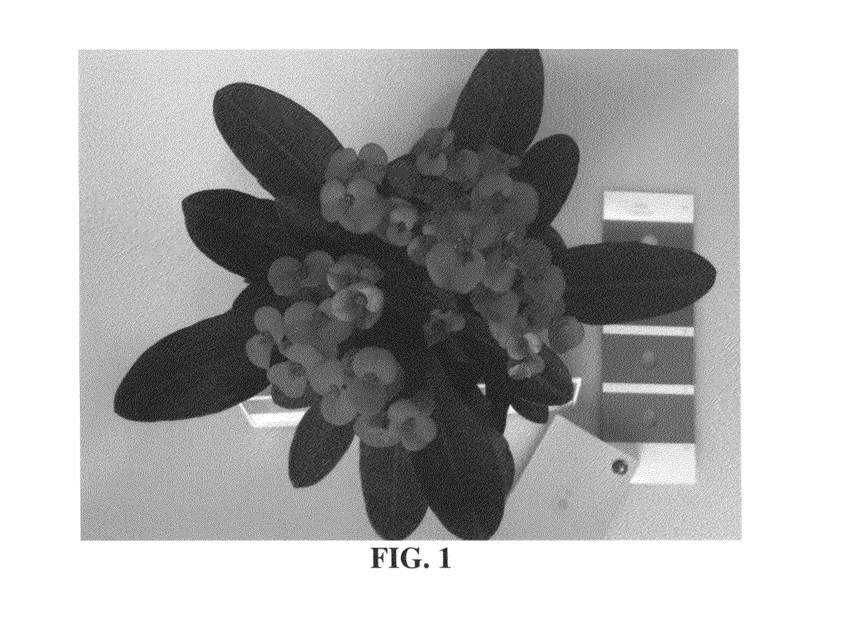

FIG. 2 shows another top view of a typical flowering plant of ‘12-01-ZEU’ as grown in an 11 cm pot (photographed under different light conditions from FIG. 1).

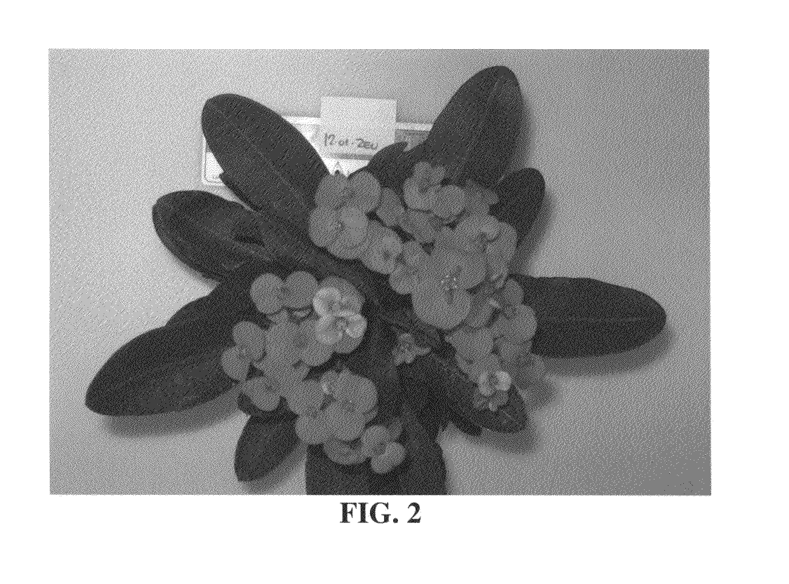

FIG. 3 shows a side view of a typical flowering plant of ‘12-01-ZEU’ as grown in an 11 cm pot.

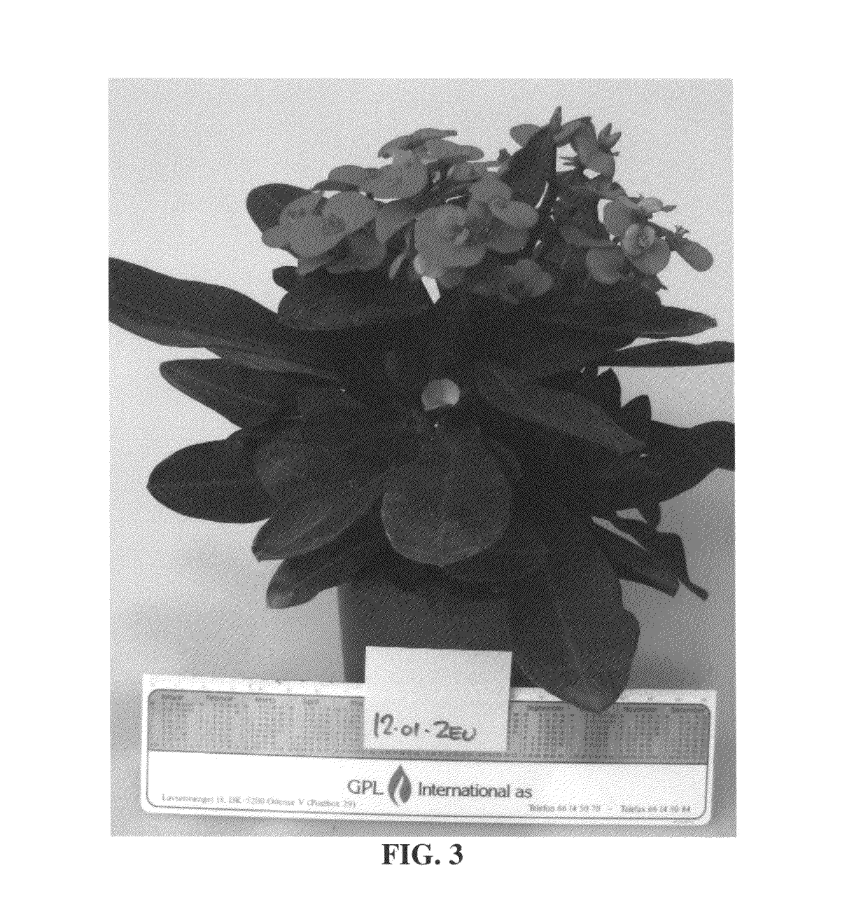

FIG. 4 shows a side view of a typical flowering plant of ‘12-01-ZEU’ as grown in an 11 cm pot (photographed under different light conditions from FIG. 3).

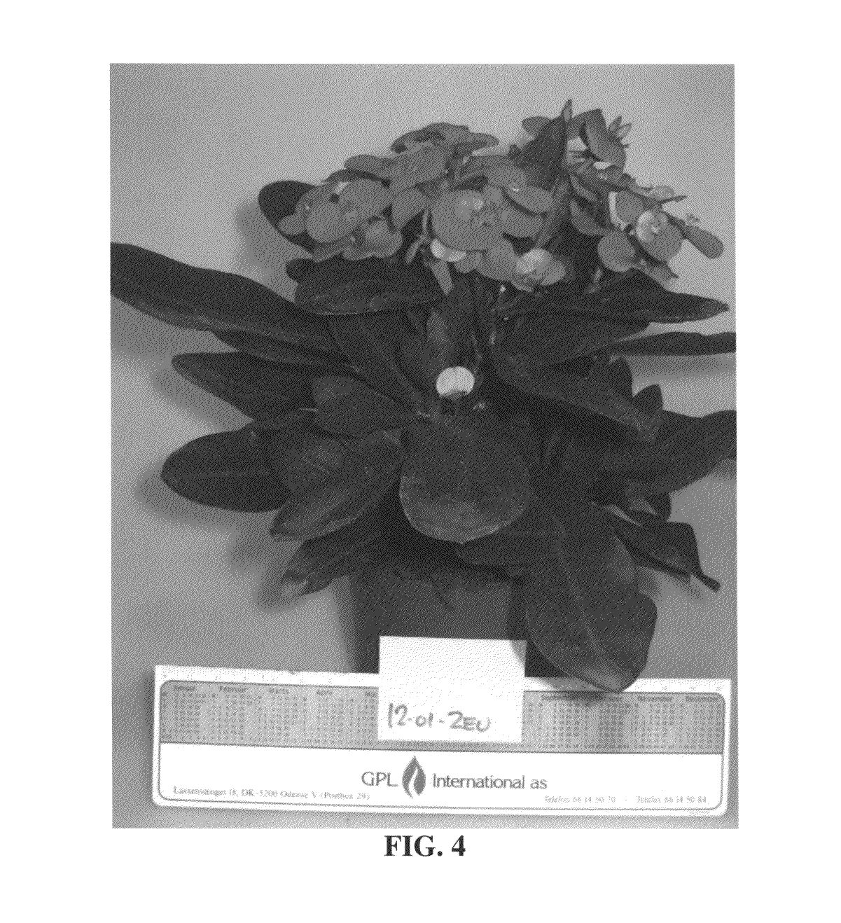

FIG. 5 is a close up of the typical flowers of ‘12-01-ZEU.’

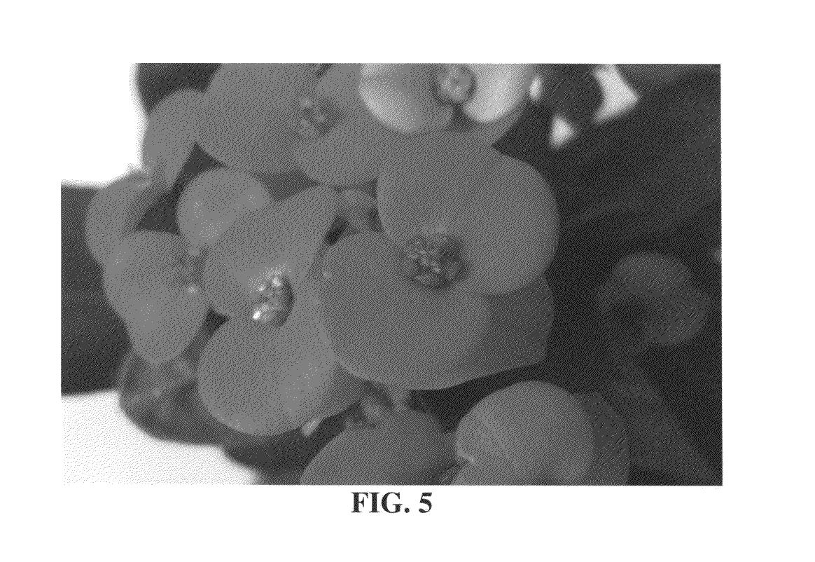

### DETAILED BOTANICAL DESCRIPTION

Plants of the new cultivar ‘12-01-ZEU’ have not been observed under all possible environmental conditions. The phenotype may vary somewhat with variations in environment such as temperature, light intensity, day length, and fertility level without, however, any variance in genotype.

The aforementioned photographs, together with the following observations, measurements and values describe plants of the new cultivar ‘12-01-ZEU’ as grown in Hjortebjergvej, Denmark, under conditions which closely approximate those generally used in commercial practice. Plants of ‘12-01-ZEU’ were grown under the temperature range of about 20-22° C. for a day length of about 12 hours. Artificial light of intensity of 50 Watt/m² was used when the intensity of the day light was less than 40 Watt/m². Water with an electrical conductivity of about 1.6 ds/m (deciSiemens/meter) was used for irrigation. All plants were grown in 11 cm pot in a glass greenhouse.

Color observations were taken under natural light, and the color references are made to the R.H.S. Color Chart of The Royal Horticulture Society of London, except where general color terms of ordinary significance are used.
Botanical classification: Euphorbia milii Desmoul.
Parentage:
   Female parent.—Unnamed seedling plant of Euphorbia milii.
   Male parent.—Unnamed seedling plant of Euphorbia milii.
Propagation:
   Type cutting.—Terminal vegetative cuttings taken from plants.
   Time to initiate roots.—about 3 weeks.
   Root description.—fibrous.
   Root color.—white.
Plant description:
   Form.—compact.
   Crop time.—about 15 weeks.
   Vigor.—medium.
   Plant size.—about 20-22 cm from soil level to top of plant plane.
   Foliage description.—leaves alternate, single, lancet shaped, about 5-6 cm in length and about 1.5-2 cm in width, upper side color being dark green, under side color being dark red.
Flower description:
   Flower arrangement.—as a bouquet, flower has no sepals or petals.
   Natural flowering season.—March-September.
   Flower longevity on the plant.—about 3-4 weeks.
   Quantity of flowers and buds per plant.—about 20-30.
   Rate of flower opening.—4-5 per week.
   Fragrance.—none.
   Infloresence size.—about 8-10 cm.
   Flowers.—round shape, upright aspect, diameter about 0.5 cm, height about 0.5 cm.
   Bracts.—two, opposite, partly overlapping at base, upper side color being RHS 045D.
   Glands.—5 glands.
   Peduncles.—variable length, sticky.
   Pedicels.—variable length, sticky.
Reproductive organs:
   Pollen.—brown colored.
   Gynoecium.—red-yellow color.
   Seeds/fruit.—3 seeds per fruit.
   Pest/disease tolerance/susceptibility.—no specific resistance or susceptibility was observed during cultivation, except some resistance against trips.

What is claimed is:

1. A new and distinct cultivar of Euphorbia milii plant named ‘12-01-ZEU,’ as illustrated and described herein.
